(19) United States
(12) Patent Application Publication
(10) Pub. No.: US 2018/0325655 A1
(43) Pub. Date: Nov. 15, 2018

(54) CORNEAL DEVICE INSERTERS AND METHODS OF USE

(71) Applicant: REVISION OPTICS, INC., Lake Forest, CA (US)

(72) Inventors: Nicolas ESGUERRA, Irvine, CA (US); Ross TSUKASHIMA, San Diego, CA (US); Alan Ngoc LE, Lake Forest, CA (US); Rachel Marie WONG, Costa Mesa, CA (US); Linh Hoai VU, Lake Forest, CA (US); Mark Douglas KEMPKEY, Vista, CA (US); Peter Kamil WAHBA, Aliso Viejo, CA (US)

(21) Appl. No.: 15/775,426

(22) PCT Filed: Nov. 14, 2016
(86) PCT No.: PCT/US2016/061775

§ 371 Date: May 11, 2018

Related U.S. Application Data
(60) Provisional application No. 62/254,112, filed on Nov. 11, 2015.
(60) Provisional application No. 62/302,687, filed on Mar. 2, 2016.

(51) Int. Cl. A61F 2/14

(52) U.S. Cl. CPC A61F 2/148; A61F 2/1451

(57) ABSTRACT

Corneal device inserters and positioners that include an elongate member with a distal region, the distal region including a top portion, a bottom portion, and a corneal device receiving space, the corneal device receiving space defined by the top portion, the bottom portion, and a receiving space proximal end, the distal region further comprising a slot in fluid communication with the corneal device receiving space, the slot extending through at least one of the top portion and the bottom portion, the slot having a slot proximal end disposed at least as far proximally as the receiving space proximal end.

### CROSS REFERENCE TO RELATED APPLICATIONS

This application claims priority to U.S. Provisional Application No. 62/254,112, filed Nov. 11, 2015, and to U.S. Provisional Application No. 62/302,687, filed Mar. 2, 2016, the disclosures of which are incorporated by reference herein.

This application is related to U.S. Pat. No. 8,540,727, the disclosure of which is incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Devices are known that are adapted to deliver, implant, and position ocular devices. Some devices, however, due to a variety of factors such as, without limitation, size and/or material, may benefit from simpler, more controlled, and/or more repeatable delivery processes. The disclosure herein provides devices and methods of use that can provide at least one of these advantages.

### DESCRIPTION OF FIGURES

FIGS. 1A, 1B, 1C, 1D, and 1E illustrate an exemplary inserter with a slot having a proximal end disposed at least as far proximally as an device receiving space proximal end.

FIG. 2 illustrates an exemplary pocket.

FIG. 3 illustrates a top view of an exemplary inserter with a slot.

FIG. 4 illustrates a top view of an exemplary inserter with a slot, in use with a device that has a diameter larger than a width of an device receiving space of the inserter.

FIG. 5 illustrates a top view of an exemplary inserter with a slot.

FIGS. 6A, 6B, and 6C illustrate an exemplary inserter with a proximal slot and distal slot.

FIG. 7 illustrates a sectional view of an exemplary inserter with a slot, the slot having a beveled surface.

FIGS. 8A and 8B illustrate an exemplary inserter with a slot.

FIGS. 9A and 9B illustrate an exemplary inserter with a slot.

FIGS. 10A and 10B illustrate an exemplary inserter with a slot.

FIGS. 11A and 11B illustrate an exemplary inserter with a slot.

FIGS. 12A and 12B illustrate an exemplary inserter with a plurality of slots.

FIGS. 13A and 13B illustrate an exemplary inserter with a slot.

FIGS. 14A and 14B illustrate an exemplary inserter with a slot.

### SUMMARY

The disclosure includes a corneal device inserter, comprising an elongate member comprising a distal region, the distal region including a top portion, a bottom portion, and a corneal device receiving space, the corneal device receiving space defined by the top portion, the bottom portion, and a receiving space proximal end, the distal region further comprising a slot in fluid communication with the corneal device receiving space, the slot extending through at least one of the top portion and the bottom portion, the slot having a slot proximal end disposed at least as far proximally as the receiving space proximal end.

The slot proximal end may be disposed proximal to the receiving space proximal end.

The slot may not extend to a distal end of the distal region.

The distal region may comprise a second slot extending through at least one of the top portion and the bottom portion and in fluid communication with the corneal device receiving space, the second slot extending to the distal end of the distal region.

The slot may extend to a distal end of the distal region.

The slot may be symmetric about a distal region longitudinal axis that extends along the distal region and through the distal end.

The distal region may comprise first and second arms extending to a distal end of the elongate region. First and second arms may be defined by the slot. First and second arms may be defined by a second slot different than the slot.

The slot may extend through the top portion and the bottom portion.

The slot proximal end can be between 0 mm and 5 mm proximal to the receiving space proximal end.

The slot may have a length from 0.5 mm and 5 mm.

The slot may have a length from 2 mm to 4 mm.

The slot may have a greatest width dimension from 0.2 mm to 2 mm.

The slot may have a greatest width dimension from 0.2 mm to 0.7 mm.

A width of the slot relative to an overall width of a distal region of the inserter can be from 5% to 40%, optionally 10% to 30%, such as 20%.

A distance between a distal end of the slot and a proximal end of the receiving space can be from 0.1 mm to 0.5 mm, optionally 0.2 mm to 0.5 mm, optionally 0.3 mm to 0.45 mm.

A distance from a distal end of the inserter to a distal end of the slot can be from 0.5 mm to 2.5 mm, optionally 1 mm to 2.25 mm.

A length of a the slot can be from 0.2 mm to 1.8 mm, optionally 0.4 mm to 1.5 mm, optionally 0.5 mm to 1.3 mm.

A width of a the slot can be from 0.2 mm to 1.5 mm, optionally 0.2 mm to 1 mm, optionally 0.2 mm to 0.7 mm.

A length of a bridge between the slot and an optional distal slot may be 0.2 mm to 1.2 mm, such as 0.5 mm to 1 mm.

A height of the receiving space can be 0.075 mm to 0.225 mm, such as 0.1 mm to 0.2 mm.

A length of the receiving space can be from 1.25 mm to 3.5 mm, such as 1.5 mm to 3 mm.

The slot may have a uniform width over at least 70% of its length.

The slot may not have a uniform width over at least 70% of its length.

The inserter may have a corneal device positioned at least partially within the corneal device receiving space, optionally in a substantially non-deformed configuration. The corneal device may be selected from the group consisting of a corneal device with a meniscus configuration and a corneal device with a pinhole configuration. The corneal device may have a water content of at least 50%, such as at least 70%.

The distal region may not be adapted to be reconfigured.

The slot and receiving space may be sized and positioned such that a corneal device can be delivered into a corneal pocket from the receiving space by proximally withdrawing the inserter, and without an active fluid delivery step and without the need for a separate delivery tool, such as a Sinskey hook.

The receiving space may extend from a first side of the distal region to a second side of the distal region.

A surface of the slot, optionally a distal surface, may be beveled.

The slot may comprise a first section and a second section, the first section having a width different than a width of the second section.

The disclosure includes a method of releasing a conical device onto corneal tissue, comprising positioning an inserter adjacent corneal tissue, optionally within a corneal pocket, wherein the inserter comprises a distal region, the distal region including a top portion, a bottom portion, and a corneal device receiving space, the corneal device receiving space defined by the top portion, the bottom portion, and a receiving space proximal end, the distal region further comprising a slot in fluid communication with the corneal device receiving space, the slot extending through at least one of the top portion and the bottom portion; and proximally retracting the inserter to release a corneal device from the receiving space, wherein the method does not require an active fluid delivery step, the use of a plunging mechanism, or reconfiguring the distal region of the inserter to release the corneal device from the receiving space.

### DETAILED DESCRIPTION

One technique for positioning a corneal device (e.g. a corneal inlay or onlay) within or on corneal tissue (which may be referred to herein as “insertion,” “delivering,” or “positioning”) is to position the device into a pocket previously created with, for example, a femtosecond laser. Corneal devices can also be positioned onto corneal tissue (e.g., stromal tissue) after a flap has been created and folded back to expose a corneal bed. Some pocket delivery methods can include one or more of active fluid delivery, a plunger mechanism being activated, or a distal end being reconfigured, to cause, or at least assist with, the deployment of the corneal device from an insertion device. It may be desirable to deliver corneal devices into pockets without one or more of these delivery assistance techniques. By not including one or more of these assistance techniques, the delivery process may be simplified, and, in the case of active fluid delivery, it may avoid delivering too much fluid into the cornea during the procedure. A merely exemplary aspect of some of the embodiments herein is that the inserters can be used to advance a corneal device into a corneal pocket and to deploy the device onto corneal tissue from the inserter device without one or more of these assistance techniques, and in some cases, without any of them. In some embodiments, however, the method may include optionally using a separate delivery tool such as Sinskey hook, examples of which are described herein.

One aspect of the delivery processes described herein is that it, when used with some hydrogel devices with relatively high water content (e.g., above 50%), it is preferred, and perhaps important, to have a “wet” environment into which to deliver the device. The methods of delivery herein thus preferably, depending on the corneal device, include steps to ensure sufficient hydration in the pocket. For example, irrigating the pocket with saline prior to insertion can help ensure sufficient irrigation in the pocket. Additionally, adding saline to the inserter itself prior to delivery can help ensure there is sufficient hydration. It is also preferred to remove any air bubbles that may be present.

The disclosure is related to the disclosure in U.S. Pat. No. 8,540,727, which is incorporated by reference herein. Suitable disclosure from U.S. Pat. No. 8,540,727 may be incorporated to the disclosure herein, such as materials used, alternative designs, and steps in methods of use.

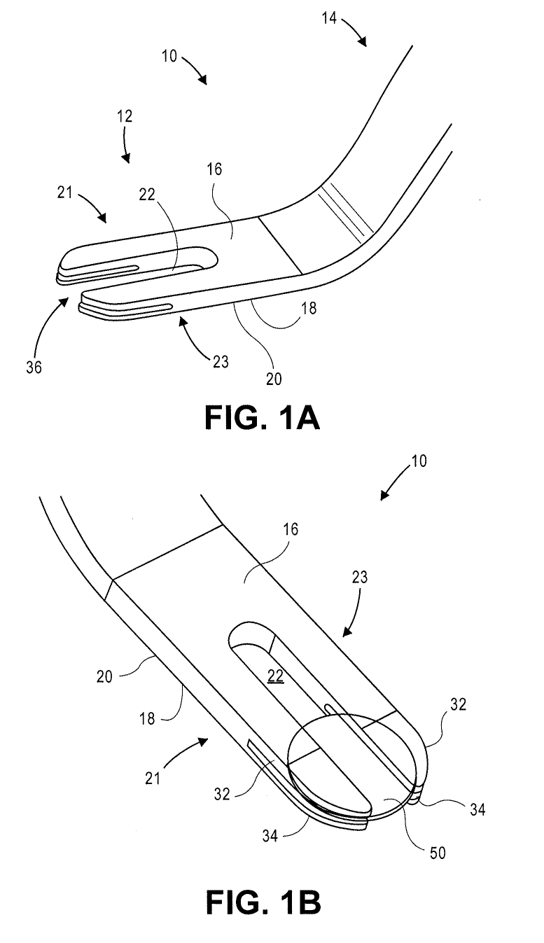

FIG. 1A illustrates a portion of an example of a corneal device inserter, and FIGS. 1B-1E illustrate an exemplary corneal device that may be delivered using the inserter from FIG. 1A. FIG. 1A illustrates inserter 10, which includes distal portion 12 (which may be referred to herein as a “distal region”) and proximal portion 14, optionally integrally formed or detachable. Distal portion 12 includes a top (or anterior) surface 16, a bottom (or posterior) surface 18, and sidewalls 20 on both sides. Surfaces 16 and 18 are shown as flat, but one or both may be configured with slight curves to them in the distal portion 12. Distal portion 12 also includes first arm 21 and second arm 23 extending on either side of distal portion 12. The inner walls of arms 21 and 23 at least partially define slot or recess 22 (better shown in FIG. 1B), with slot 22 extending along a portion of distal portion 12. Slot 22 is an example of a “space,” a volume,” and a “recess.” Slot 22 extends through the top and bottom surfaces 16 and 18 of the distal portion. Slot 22 can be created in the distal portion 12 using, for example, an end mill. In some manufacturing techniques, creating slot 22 creates first and second arms 21 and 23 in the distal portion. The slot can be created using other techniques as well. First and second arms 21 and 23 each include top extensions 32 and bottom extensions 34 (see FIG. 1B), which define the top and bottom of device receiving volume, or space, 36. The device receiving spaces (part of the overall receiving space 36) between the top and bottom extensions have, in this embodiment, the same dimensions, and are longer (measured along length of distal portion 12) than they are tall (measured from top 16 to bottom surface 18). Slot 22 extends further proximally than the top and bottom extensions of arms 21 and 23. In this embodiment, slot 22 is longer than the top and bottom extensions on arms 21 and 23. In this embodiment, the top and bottom extensions 32, and 34, respectively, of both arms are parallel with one another, and are not adapted to be moved relative to one another. The top portion of the distal region, in this embodiment, comprises top extensions 32, and the bottom portion of the distal region, in this embodiment, comprises bottom extensions 34.

FIGS. 1B-1E illustrate different views of inserter 10, and an exemplary device 50 that is at least partially disposed in receiving space 36, wherein device 50 can be delivered onto corneal tissue using inserter 10. FIGS. 1B-1E illustrate device 50 disposed between top extensions 32 and bottom extensions 34 of arms 21 and 23, part of the corneal device receiving space. Device 50 can be stored in this position, it can be positioned in distal region 12 of the inserter just prior to insertion, or it can be placed in this position at any other time. Because slot 22 extends further proximally than the proximal end PE of device receiving space 36, when device 50 is positioned in the distal region 12 in between the extensions on arms 21 and 23, there is a length of slot 22 behind, or proximal to, device 50. This open space behind the device (which may be referred to herein as a “space,” or “void”) can have significance in delivering device 50.

The inserter 10 shown in FIGS. 1A-1E was initially prototyped as a way to deliver device 50 into a corneal pocket without relying on active fluid delivery to deploy the device from the distal region of the inserter and into the pocket. Designing slot 22 with a relatively longer length, relative to this particular device 50, was initially contemplated as part of a way to allow a separate tool (e.g., a Sinskey hook) to be pushed gently downward on the cornea to assist in removing the device from the device receiving space at the distal region of the inserter. Specifically, it was contemplated that after the inserter (within device retained at the holding space) was inserted into the pocket channel and into the pocket, a separate tool could then be urged gently downward generally in alignment with slot 22, and perhaps with slightly greater pressure behind the device at the slot location. It was contemplated that, during the next step of retracting the inserter, the separate tool would be able to apply a slight force to help retain the device at the desired location within the pocket without being pulled back out of the pocket along with the inserter.

An unexpected result occurred during an initial testing of the inserter. After the inserter and device were advanced into the pocket, the inserter was then retracted back out of the pocket without applying any force on the cornea with a separate tool. The device unexpectedly deployed from the distal region of the inserter and stayed in the pocket in the desired location. The device was inserted into a pocket without any active fluid delivery, without the need of a separate tool to disassociate the device from the distal region of the inserter, without using a plunger mechanism (which can be common in intraocular lens delivery methods), and without reconfiguring the distal region of the inserter. The unexpected result was a dramatically simpler procedure and the device was delivered to the desired location within the pocket. An optional step is to purge the pocket with saline prior to insertion, which may help with the passive fluid assist.

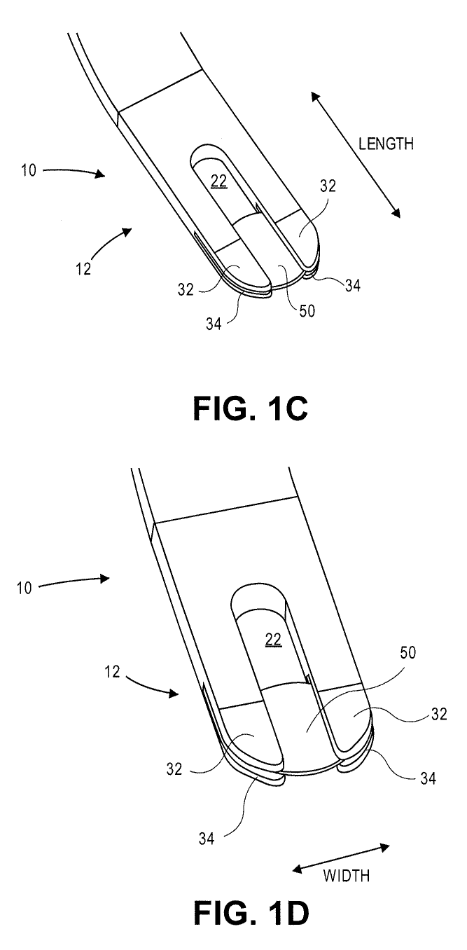

It is of note that the tested corneal device was a corneal inlay with a diameter between 1.5 mm and 3 mm (in a testing it was about 2.0 mm), a central thickness between 20 microns and 50 microns (in a testing it was about 32 microns) and made of a hydrogel material with a relatively high water content of at least 50% (e.g., nearly 80% water). The pocket created in the test eye has dimensions in millimeters illustrated in FIG. 2, and the pocket depth was about 180 to about 240 microns. The pocket was created with a femtosecond laser. In some embodiments the inserters herein can be used to deliver any type of corneal device with a water content of at least 50%. In some embodiments, however, the inserters may be successful in delivering corneal devices whose water content is less than 50%.

Without wishing to be bound by a particular theory, a possible contributing mechanism of action is that fluid and/or air may fill up (occupy) the empty slot space behind the device during the delivery process (e.g., as the inserter and device are advanced into the pocket). For example, fluid can be retained in the slot behind the device due to surface tension. As the inserter is retracted, the fluid and/or air that is within the slot may help prevent the device from being retained by the distal region. That is, the fluid and/or air may help disassociate the device from the distal region (deliver the device). This process may be considered a type of passive fluid assistance for insertion, but again the retraction step does not require an active fluid delivery towards the distal region of the inserter. The fluid in the slot can help immobilize the device as the inserter is retracted. The fluid in the slot may also help break the adhesion forces between the device and the inserter, which can cause the device to remain in place in the pocket as the inserter is retracted. Again, without wishing to be bound by theory, one possible contributing mechanism of action (although to date no evidence of this theory has been seen) is that the slot may also act as an air channel to allow fluid inside the corneal pocket to not be disturbed. The process may be analogized to putting a finger on the end of a straw in a cup of water and lifting the straw up. Keeping the finger on the end of the straw as the straw is lifted up (the fluid moves with the straw) can be compared to an inserter without an adequate slot. An inserter with an adequate slot, however (such as those described herein), can be compared to removing the finger from the end of straw and then lifting up on the straw (the fluid does not move with the straw). Again, without wishing to be bound by theory, a possible contributing mechanism of action is that the corneal tissue may conform at least somewhat to the slot cavity, and essentially “grab” the corneal device from the anterior and posterior sides during delivery. By “grabbing” the device, the corneal tissue can be thought of as holding onto the device and preventing it from being retracted with the inserter. Thinner (measured as height herein) distal regions may in some instances enhance the tissue's ability to “grab” onto and “hold” the device during the delivery process. Varying the thicknesses (height) of the inserter distal region may thus be beneficial in controlling one or more aspects of the deployment process. Some device designs/configurations/sizes may benefit from thinner or thicker distal regions, similar to varying the length and/or width of the inserter slot as described herein.

While one particular inserter design is shown in FIGS. 1A-1E, it is understood, as described below, that other configurations may also be used in the same or similar manner, particularly designs that may be used with other device sizes and configurations.

FIG. 1C illustrate an exemplary length dimension of slot 22, which may have a proximal end is that is disposed at least as far proximally as the receiving space proximal end. In some embodiments slot 22 has a length between 1 mm and 7 mm, measured from the distal end of the inserter. In some exemplary embodiments the slot has a length between 1.5 mm and 6 mm, between 1.75 mm and 6 mm, between 1.75 mm and 5 mm, between 2 mm and 7 mm, between 2 mm and 6 mm, 2 mm and 5.5 mm, 2 mm and 5.0 mm, between 2 mm and 4.5 mm, 2 mm and 4 mm, between 2.5 mm and 7 mm, between 2.5 mm and 6 mm, between 2.5 mm and 5.5 mm, between 2.5 mm and 5.0 mm, between 2.5 mm and 4.5 mm, between 2.5 mm and 4.0 mm, between 2.5 mm and 3.5 mm, between 3.0 mm and 7 mm, between 3.0 mm and 6 mm, between 3.0 mm and 5.5 mm, between 3.0 mm and 5.0 mm, between 3.0 mm and 4.5 mm, between 3.0 mm and 4.0 mm, between 3.5 mm and 7.0 mm, between 3.5 mm and 6.0 mm, between 3.5 mm and 5.5 mm, between 3.5 mm and 5.0 mm, between 3.5 mm and 4.5 mm, between 3.5 mm and 4.0 mm.

In some embodiments slot 22 has a length of about 1.75 mm, about 2 mm, about 2.25 mm, about 2.5 mm, about 2.75 mm, about 3 mm, about 3.25 mm, about 3.5 mm, about 3.75 mm, about 4.0 mm, about 4.25 mm, about 4.5 mm, about 5.0 mm, about 5.25 mm, about 5.5 mm, about 5.75 mm, about 6.0 mm, about 6.25 mm, about 6.5 mm, about 6.75 mm, about 7.0 mm, or about 7.25 mm.

In some embodiments, however, the slot is longer than described above. For example, it may be beneficial to have a slot with a length greater than 7 mm. With any of the lower end of ranges herein, the upper end of the range can be 8 mm, 9 mm, 10 mm, or even greater. It is thus understood that a range that includes a lower end but not an upper end (e.g., at least 1 mm) is not indefinite in that the lower end of the range may be more important than the upper end, in which case not including an upper end of the range means that it can be any suitable upper limit, depending on the type of inserter being used.

The exemplary slot lengths listed above are different than, for example, the slot lengths shown in U.S. Pat. No. 8,540,727, co-owned by Applicant. For example, the slots in U.S. Pat. No. 8,540,727 are shown as much shorter than the device in the device holding space, and an exemplary diameter of the device therein is provided as about 1.5 mm. The slot lengths provided herein thus are described as being longer than the slot lengths in U.S. Pat. No. 8,540,727, and the reason for the increase in length is set forth herein. The slot lengths in U.S. Pat. No. 8,540,727 were apparently as long as needed to allow a tool such as Sinskey hook to simply apply some pressure onto the device through the top slot during delivery, and were not described as having lengths as set forth herein, nor were they described as providing the advantages of the slots and methods of delivery herein. The ranges and examples of slot lengths herein is thus intentionally described to differentiate over slot lengths such as in U.S. Pat. No. 8,540,727, and are not simply arbitrarily chosen lengths. The slot length can vary and still be used in the methods described herein. In one merely exemplary testing of the inserter tool and the device shown in FIGS. 1B-1E, the slot length was about 4 mm.

The slot length may vary depending on the size, configuration, and material of the corneal device that is being delivered.

As shown in the exemplary embodiment in FIGS. 1A-1E, the slot width (“w”) is constant along substantially the entire length of slot, with the exceptions being at the very ends of the slot.

In exemplary embodiments the slot width (measured in the direction shown in FIG. 1D) is between 0.25 mm and 1.75 mm, between 0.25 mm and 1.50 mm, between 0.25 mm and 1.25 mm, between 0.25 mm and 1.00 mm, between 0.25 mm and 0.75 mm, between 0.25 mm and 0.50 mm, between 0.50 mm and 1.75 mm, between 0.50 mm and 1.50 mm, between 0.50 mm and 1.25 mm, between 0.50 mm and 1.00 mm, between 0.50 mm and 0.75 mm, between 0.75 mm and 1.75 mm, between 0.75 mm and 1.50 mm, between 0.75 mm and 1.25 mm, between 0.75 mm and 1.00 mm, between 1.00 mm and 1.75 mm, between 1.00 mm and 1.50 mm, between 1.00 mm and 1.25 mm, between 1.25 mm and 1.75 mm, between 1.25 mm and 1.50 mm, or between 1.50 mm and 1.75 mm.

In some embodiments the width is about 0.25 mm, about 0.50 mm, about 0.75 mm, about 1.00 mm, about 1.25 mm, about 1.50 mm, or about 1.75 mm.

The width of a slot can vary and still allow the inserter to be used in the manner herein.

The width of a slot can be different than the ranges and examples provided herein, such as if the device size and/or configuration changes.

In alternative designs the width is not constant over substantially the entire length of the slot. For example, the distal region of a slot near the device may have a smaller width than the proximal region of the slot proximal to the device. Varying the slot width along its length may allow for more controlled and precise device delivery.

Any of the slot lengths herein (ranges or discrete numbers) can be used with any of the slot widths herein (ranges or discrete numbers).

FIG. 1E is a perspective side view of the distal region 12 of inserter 10 with corneal device 50 disposed within the receiving space, as is also shown in FIGS. 1B-1D.

In this embodiment the height “h” of device receiving space 36 is greater than the height of device 50. In this embodiment, the device is disposed in receiving space 36 and is in a substantially non-deformed, or non-stressed, configuration (i.e., the as-manufactured configuration). “Substantially,” as used in this manner indicates that there may be some incidental forces acting on the device, but none large enough to cause substantial deformation to the device. This can be beneficial in that stresses are not imposed on the device prior to insertion, which can prevent damage to the device.

In some embodiments the height of any of the receiving spaces herein (i.e., the distance between the extensions) is between 0.075 mm and 0.225 mm, between 0.075 mm and 0.200 mm, between 0.075 mm and 0.175 mm, between 0.075 mm and 0.150 mm, between 0.075 mm and 0.125 mm, between 0.075 mm and 0.100 mm, between 0.100 mm and 0.225 mm, between 0.100 mm and 0.200 mm, between 0.100 mm and 0.175 mm, between 0.100 mm and 0.150 mm, between 0.100 mm and 0.125 mm, between 0.125 mm and 0.225 mm, between 0.125 mm and 0.200 mm, between 0.125 mm and 0.175 mm, between 0.125 mm and 0.150 mm, between 0.150 mm and 0.225 mm, between 0.150 mm and 0.200 mm, between 0.150 mm and 0.175 mm, between 0.175 mm and 0.225 mm, between 0.175 mm and 0.200 mm, or between 0.200 mm and 0.225 mm. The height of the receiving space may vary depending on the size and/or configuration of the device being delivered, and is not limited the ranges herein.

In some embodiments the corneal device has a height (which may be different than the central thickness depending on the shape of the device) that is about between about 0.050 mm and 0.100 mm, such as about 0.075 mm.

In this embodiment the height of the receiving space is greater than the height of the corneal device, and is about two times the height.

In some embodiments the height of the distal region, or at least the distal end of the distal region, is between 0.25 mm and 1.25 mm, such as between 0.25 mm and 1.00 m, between 0.25 mm and 0.75 mm, such as about 0.50 mm.

The lengths, widths, and heights described herein may be combined in any desired manner.

In some embodiments in which a hydrogel corneal device is delivered with the inserter, the device may be retained at the distal region of the inserter at least partially by surface tension.

Possible contributing mechanisms of actions for delivering the device have been described herein. It may be possible to vary the length of the slot and still create enough space so that the device is properly deployed as the inserter is withdrawn. In some embodiments the slot may be just slightly longer than the length (or diameter) of the device when disposed in the receiving space. For example, the length of the slot behind the device may be smaller than shown in FIGS. 1A-1E, and the delivery process may still deliver the device without or more of the delivery assistance techniques described herein. Alternatively, the slot may be smaller, and it may be helpful to use a separate tool to assist with the delivery. Alternatively still, a separate tool may be used to gently urge downward on corneal tissue as an assurance technique, even if it is not a necessary step in the procedure.

The disclosure herein includes inserters in which a slot proximal end is disposed at least as far proximally as the device receiving space proximal end, and optionally further proximally than the device receiving space proximal end. For example, in the embodiment of the inserter shown in FIGS. 1A-1E, slot 22 extends further proximally than the device receiving space. This creates an open space that is proximal to the receiving space and the eventual device disposed at the receiving space, and is believed to assist in the passive delivery of the devices as set forth herein.

In some embodiments the slot may not extend all the way through top surface 16 or bottom surface 18. For example, it may only be needed to have slot extend through one of the top and bottom surfaces.

It is also conceivable that in some embodiments, if the width of the slot is great enough, the slot may not need to extend back proximally as far, and may in fact have a proximal end that extends only as far as the device receiving space proximal end.

The disclosure herein also includes systems that include the inserter and the device positioned with the inserter. The device can be positioned at a distal region of the inserter (as shown in FIGS. 1B-1E) any time before the delivery is initiated. Exemplary systems include inserters and corneal device in which an inserter slot extends at least as far proximally as a device receiving space, and optionally further proximally than a proximal end of the device receiving space. For example, in FIG. 1B, device receiving space 36, which is configured to retain device 50, is just slightly longer than the diameter of the device, and slot 22 extends further proximally than the receiving space, as well as the device.

It has also been shown that inserter 10 can be used to position the device 50 on a stromal bed after a flap has been created and lifted. Inserter 10 can thus be used to deliver an device after flap or pocket creation. When placed after a flap has been lifted, the inserter may be able to deliver the device without one or more of the assistance techniques described herein (e.g., active fluid delivery).

In some embodiments the length of the slot is greater than the length of the device receiving space. In some embodiments the slot length is at least 105% of the device receiving space length, at least 110% of the device receiving space length, at least 115% of the device receiving space length, at least 120% of the device receiving space length, at least 125% of the device receiving space length, at least 130% of the device receiving space length, at least 135% of the device receiving space length, at least 140% of the device receiving space length, at least 145% of the device receiving space length, at least 150% of the device receiving space length, at least 155% of the device receiving space length, at least 160% of the device receiving space length, at least 165% of the device receiving space length, at least 170% of the device receiving space length, at least 175% of the device receiving space length, at least 180% of the device receiving space length, at least 185% of the device receiving space length, at least 190% of the device receiving space length, at least 195% of the device receiving space length, or at least 200% of the device receiving space length.

In systems herein that include a corneal device within the receiving space of the inserter, the slot can extend at least as far proximally as the proximal end of the device when positioned in the device receiving space.

The inserters herein may be used with caps that cover the distal region, such as caps described in U.S. Pat. No. 8,540,727 and U.S. Pub. No. 2013/0023892.

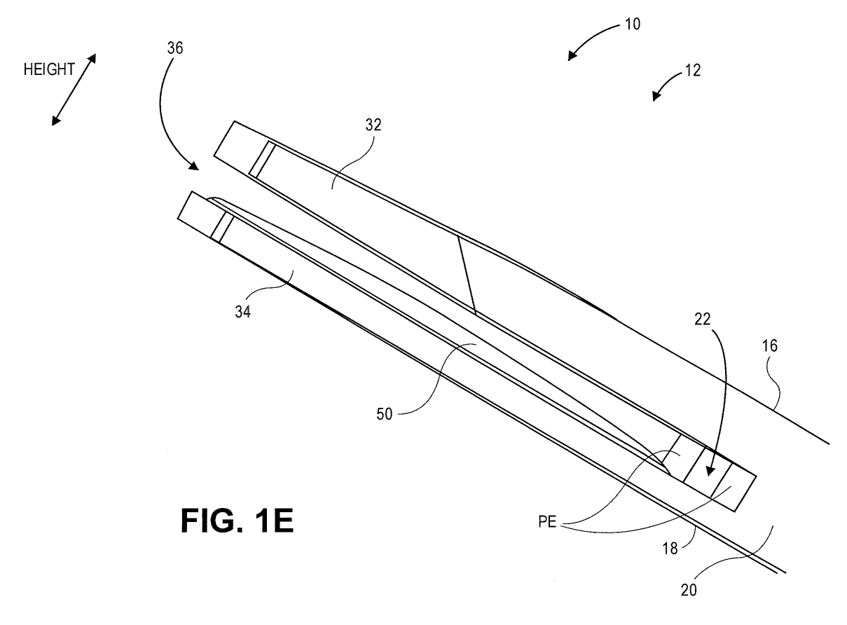

In some embodiments, one or more inserter slots do not extend all of the way to the distal end of the inserter. For example, FIG. 3 illustrates a top view of an alternative distal region of an inserter, in which slot 62 is still in fluid communication with the device receiving space, but does not extend as far as the distal end 64. The inserter shown in FIG. 3 can be the same as the inserter in FIGS. 1A-1E, with the exception of the slot extending to the distal end. The inserter in FIG. 3 thus does not have extensions 32 and 34, but still has a top portion and a bottom portion, which partially define the device receiving space. Slot 62 extends through the top and bottom surfaces, as in the embodiment in FIGS. 1A-1E. An exemplary corneal device and device receiving space proximal end are both shown in phantom in the FIG. 3 top view. This is an example of a slot proximal end disposed further proximally than a device receiving space proximal end, even if the slot does not extend to the distal end of the inserter. In some embodiments, any of the slots herein can have a length (from distal end to proximal end in a top view) that is actually shorter that the length of the device receiving space. In FIG. 3, slot 62 can still cause the device to be deployed in the pocket as the inserter is retracted without assistance techniques described herein. In any embodiment wherein one or more slots do not extend to the distal end (e.g., FIG. 3), the slot can still have any of the dimensions described herein (e.g., length, width), and can have any position relative to other inserter components described herein.

The slot can have a length that is the same as or shorter than the device receiving space, and can be shorter than the device. For example, the inserter distal end may be configured so that a slot length is adequate to allow the device to be delivered into a pocket as described herein. A shorter slot may create more stability for the corneal device during, for example, packaging. The slot width can be varied along with slot length to create a slot space that allows the device to be properly delivered.

In some embodiments the slot length varies when comparing the slot length along the top and bottom surfaces. For example, the slot may extend to the end in one of the top and bottom surfaces, but not the other. For example, the slot may extend to the end of the top surface but not the end of the bottom surface. The slot may not even extend through any of one of the surfaces, but it may extend through at least a portion of the other surface.

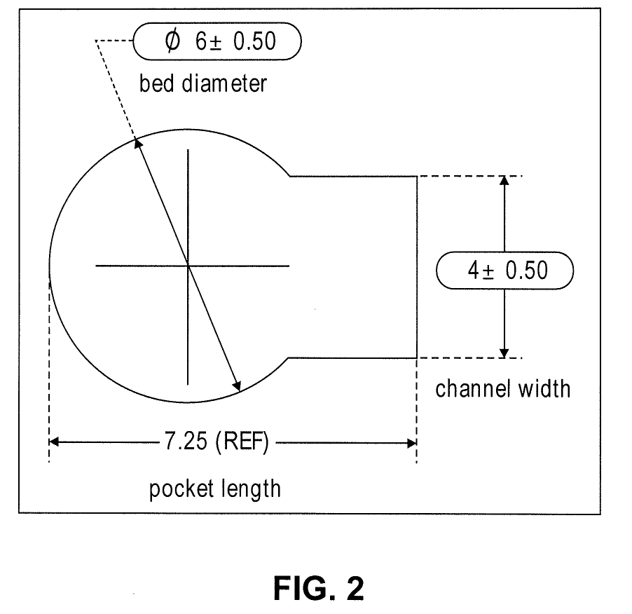

FIG. 4 illustrates a top view of an inserter that can be the same as shown in FIGS. 1A-1E, but the device is pinhole device 70. Slot 72 could also be configured like slot 62 in FIG. 3. Device 70 is configured as a pinhole device, and has a diameter larger than the width of the device receiving space of the inserter. In some embodiments the device has a diameter between about 3 mm about 6 mm. The inserter can be as described above, and in this embodiment the device extends outside of the boundary of the distal region of the inserter, in a top view. While there may be drawbacks to this type of system, it can be used. The receiving space proximal end is also shown in the phantom straight lines.

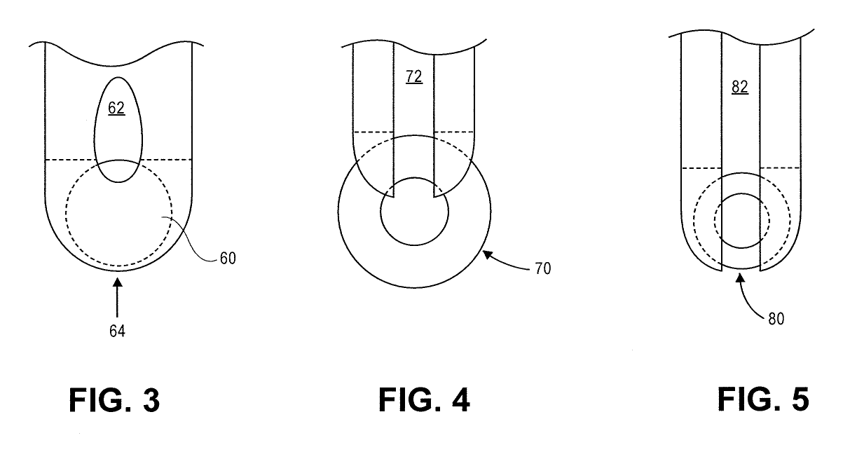

FIG. 5 is a top view illustrating an inserter (which can be the same as the inserter in FIGS. 1A-1E) and pinhole corneal device 80, wherein the pinhole device is not disposed “outside” of the boundaries of the device receiving space (unlike FIG. 4). In some embodiments the device has a diameter between about 3 mm and about 6 mm, and thus the dimensions of the inserter distal region would be modified accordingly. Any of the general considerations set forth herein, however, could still apply to the design. For example, the slot 82 proximal end is disposed further proximally than the proximal end of device receiving space. The device, such as devices 70 and 80, need not have relatively high water content, and need not be a hydrogel.

In some embodiments herein, the extensions of each arm (when the embodiment includes them, unlike FIG. 3) are not configured to be moved apart from each other. In some embodiments, however, the inserter can be adapted so that one or both extensions, or top and bottom portions if the inserter does not have any extensions, can be caused to move relative to the other (e.g., up/down, back/forth), which may assist in freeing the device from the receiving space. For example, inserter designs described in US Pub No 2011/0172675 may be incorporated into the inserters herein.

The top and bottom surfaces and portions of the distal regions of the inserters can be flat as shown herein, or may have a slight curve to them (when viewed from the side). The top surface (such as top surface 16) of the top portion may be slightly beveled downward toward the distal end, which may make it easier to advance the inserter.

As used herein, “slot,” when used generally, may be interchanged with “space” or “volume” unless indicated otherwise. For example, slot 22 in FIGS. 1A-1E does have a traditional slot configuration, but the slot can also be referred to as a space, volume, or recess.

In some embodiments the inserter may have more than one inserter slot (as used herein “inserter slot” is different than a “corneal device receiving slot”). For example, the inserter can have a plurality of inserter slots across the width of the inserter. In these embodiments the distal region could be similar to two forks on top of each other. There can thus be more than two sets of “extensions” or prongs in the distal region.

In some embodiments the one or more slots are not symmetrical top-to-bottom. For example, one or more of the prongs (the extensions) can also be offset from one another, in a top view of the distal region. When looking down on the inserter in a top view, one could thus see at least a portion of one or more bottom extensions in this type of non-symmetrical design. Additionally, in some embodiments the top surface has a different number of slots than the bottom surface. Alternatively, a top slot can have different dimensions and/or shape than the bottom slot. Alternatively, the inserter may have no slot on one surface and one or more slots on the other surface, or differing number of slots on the top and bottom surfaces. Alternatively, for example, the inserter could have a top and a bottom slot on different sides of the center line (e.g., top slot left of the center line of the distal region, and the bottom slot right of the center line, or vice versa). The inserter could also have multiple slots on different sides of a centerline. The offset slotted designs can be beneficial in still allowing the tissue to conform and “grab” the device as described above, but making it harder for the device to escape during storage or transport (more security during storage and/or transport).

All of the description above applies to all of the description below, including the exemplary examples below. For example, any of the dimensions described above can apply to any of the embodiments and aspects of the disclosure described below.

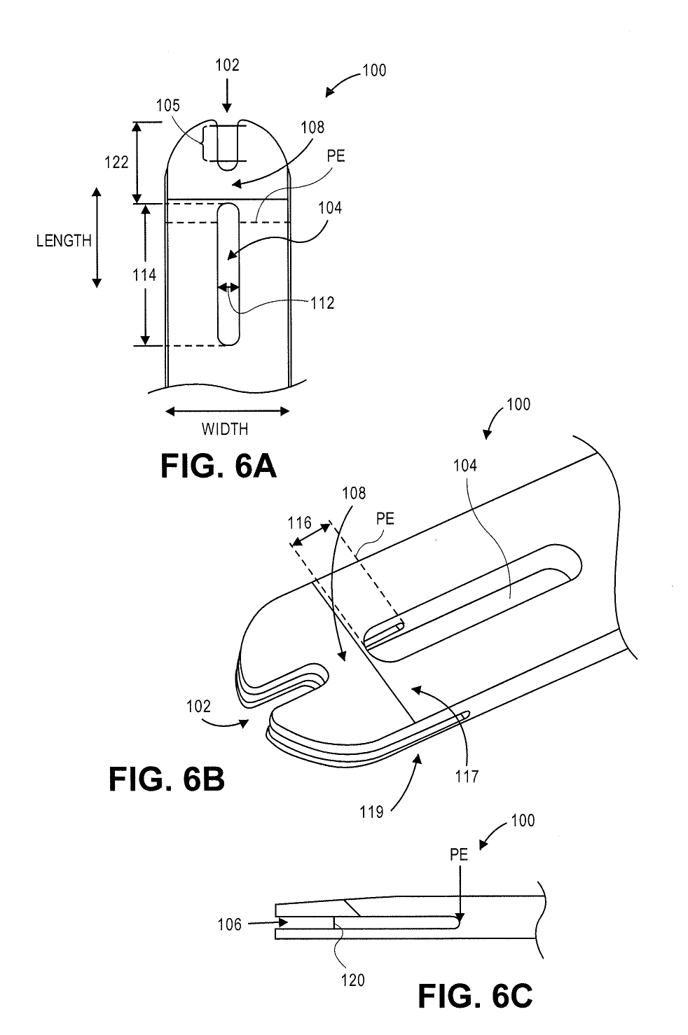

FIGS. 6A-6C illustrate a distal region of an exemplary embodiment of inserter 100 that includes an optional distal slot 102 and a proximal slot 104, both of which are in fluid communication with corneal device receiving space 106. FIG. 6A is a top view, FIG. 6B is a perspective view, and FIG. 6C is a side view. Slot 104 can be thought of as similar to slot 62 from FIG. 3, in that it does not extend to an outer edge of the distal region of the inserter (it can be considered a “window,” in a top view). Inserter 100 also includes slot 102, which can be the same or similar to any of the slots shown in U.S. Pat. No. 8,540,727, or any distal slots herein. In a top view, the slots are discrete slots (i.e., separated by inserter material), but they are both in fluidic communication via the corneal device receiving space. The two slots, in a top view, are separated by inserter bridge 108. A corneal device can be positioned in the inserter in the corneal device receiving space as shown in FIGS. 1A-1E. Slot 102 has a constant width over substantially its entire length L. As shown, the width is constant in section 105 of the slot, but slot 102 has relatively short sections distal and proximal to section 105. The width becomes slightly greater in the section distal to section 105, and decreases slightly in the section proximal to section 105. Slot 102 is an example of a slot that has a width that is constant over substantially its entire length. “Substantially” in this context means a constant width over at least 70% of its length. Inserter 100 can also include any of the suitable inserter features herein.

Without wishing to be bound by theory, proximal slot 104 may allow for fluid flow to “push” the device out of the device receiving space, as described above, when retracting the inserter during a pocket procedure. And again, without wishing to be bound by theory, an additional possible contributing factor, in addition those set forth above, for which some evidence has been seen, is that the proximal slot, when in communication with the device receiving space, helps replace the volume of the device as it is being displaced out of the receiving space. As the inserter is moved within the corneal pocket, the corneal device begins to exit the inserter. In designs without the proximal slot, the device may get sucked back into the receiving space if the inserter is pulled back too quickly. The corneal device does not, however, get pulled back into the receiving space when the proximal slot is present. This can be equated to retracting a plunger from a syringe with a very small diameter needle (the plunger being analogous to the device). If you retract the plunger too fast, the plunger will move back into position because the fluid does not have a chance to fill the newly created volume (it creates a vacuum). Adding the proximal slot to the inserter is equivalent to adding another hole to the needle and/or syringe and creating another avenue for the fluid to replace the volume, thus reducing the vacuum created by the moving plunger (in this analogy, the corneal device).

Proximal slot 104 has a constant width 112 along substantially its entire length, and a length 114 from distal to proximal end. The proximal end “PE” of the corneal device receiving space is also shown in dashed in the top view of FIG. 6A, the perspective view of FIG. 6B, and the side view of FIG. 6C. The distance between the distal end of slot 104 and the proximal end PE of the corneal device receiving space is labeled as distance 116. In this embodiment, the distal end of slot 104 is distal to the proximal end PE of the corneal device receiving space. FIG. 6C identifies height 120 of the corneal device receiving space. Inserter 100 includes top portion 117 and bottom portion 119, which can be thought of in a similar to the top and bottom portions in FIGS. 1A-1E. Inserter 100 also has first and second arms and top extensions and bottom extensions, similar to FIGS. 1A-1E. In FIGS. 1A-1E, however, the inserter does not have a bridge, such as bridge 108.

In some embodiments, length 114 of proximal slot 104 is from 0.5 mm to 5 mm, such as from 2 mm to 4 mm, such as from 2.8 mm to 3.1 mm.

In some embodiments, width 112 of proximal slot 104 is from 0.2 mm to 1.5 mm, such as from 0.2 mm to 1 mm, such as 0.2 mm to 0.7 mm, such as 0.3 mm to 0.6 mm, such as 0.4 mm to 0.5 mm.

In some embodiments, the width 112 of slot 104 relative to the overall width of the inserter distal region is from 5%-80%, such as from 5% to 50%, such as 5% to 40%, such as 5% to 30%, such as 10% to 30%, such as 20%.

In some embodiments, distance 116 between the distal end of slot 104 and the proximal end PE of the device receiving space can be 0.1 mm to 0.5 mm, such as 0.2 mm to 0.5 mm, such 0.3 mm to 0.45 mm.

In some embodiments, the distance 122 (see FIG. 6A) from the distal end of the inserter to the distal end of slot 104 may be from 0.5 mm to 2.5 mm, such as from 1 mm to 2.25 mm, such as from 1.5 mm to 2 mm, such as 1.8 mm.

In some embodiments, the length of slot 102 can be from 0.2 mm to 1.8 mm, such as 0.4 mm to 1.5 mm, such as 0.5 mm to 1.3, such as 0.9 mm.

In some embodiments, the width of slot 102 is from 0.2 mm to 1.5 mm, such as from 0.2 mm to 1 mm, such as 0.2 mm to 0.7 mm, such as 0.3 mm to 0.6 mm, such as 0.4 mm to 0.5 mm.

In some embodiments, the length of bridge 108 is 0.2 mm to 1.2 mm, such as 0.5 mm to 1 mm, such as 0.94 mm.

In some embodiments, the height 120 of the device receiving space is from 0.075 mm to 0.225 mm, such as 0.1 mm to 0.2 mm, such as 0.15 mm.

In some embodiments, the length of device receiving space 106, measured from the distal end of the inserter to the proximal end PE of the device receiving space 106, is from 1.25 mm to 3.5 mm, and this length can depend on the size of the device being deviceed. The length can be greater than 3.5 mm for larger devices, for example. In some embodiments the length is 1.5 mm to 3 mm, such as 2.2 mm.

The embodiment in FIGS. 6A-6C is an example of an inserter with an inserter slot, is an example of an inserter with an inserter slot extending through at least one of top and bottom surface of a distal region of the inserter, is an example of an inserter with an inserter slot, wherein at least a least a portion of the inserter slot is proximal to, or behind, a proximal end of an inserter device receiving space, is an example of an inserter with an inserter slot in fluidic communication with an inserter device receiving space, is an example of an inserter with an inserter slot that has a constant width along substantially its entire length (both slots 102 and 104 have constant widths over substantially their entire lengths; and either or both could be modified to have a constant width along the entire length), is an example of an inserter with a plurality of inserter slots, and is an example of an inserter slot (the proximal slot) that does not extend all the way to an outer edge of a distal region of the inserter.

The inserter in FIGS. 6A-6C is an example of a corneal device inserter, comprising an elongate member comprising a distal region, the distal region including a top portion, a bottom portion, and a corneal device receiving space, the corneal device receiving space defined at least partially by the top portion, the bottom portion, and a receiving space proximal end, the distal region further comprising a slot in fluid communication with the corneal device receiving space, the slot extending through at least one of the top portion and the bottom portion, the slot having a slot proximal end disposed at least as far proximally as the receiving space proximal end.

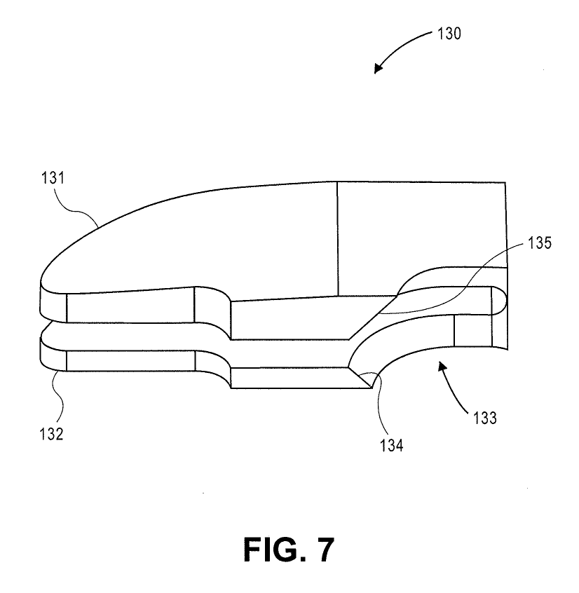

FIG. 7 is a cutout view of a portion of a distal region of exemplary inserter 130, which except for differences called out herein, can in all other ways be the same as inserter 100 from FIGS. 6A-6C, or any other inserters herein. FIG. 7 shows top extension 131 (of top portion) and bottom extension 132 (of bottom portion) of one arm on one side of the distal slot. A portion of proximal slot 133 can also be seen. One difference between inserter 100 and 130 is that proximal slot 133 in inserter 130 is beveled at the corneal device receiving area. The beveled surfaces 134 and 135 are on the top and bottom of the device receiving space, respectively, although in other embodiments only one of the top and bottom surfaces is beveled. The surfaces are beveled so that a larger area is created adjacent the device receiving space, and a smaller area is located at the top and bottom surfaces of the inserter. The beveled surfaces create ramps that are configured to direct fluid flow towards the corneal device receiving space, which aids in the device delivery from the inserter during a pocket delivery procedure. Any of the surfaces herein can be beveled.

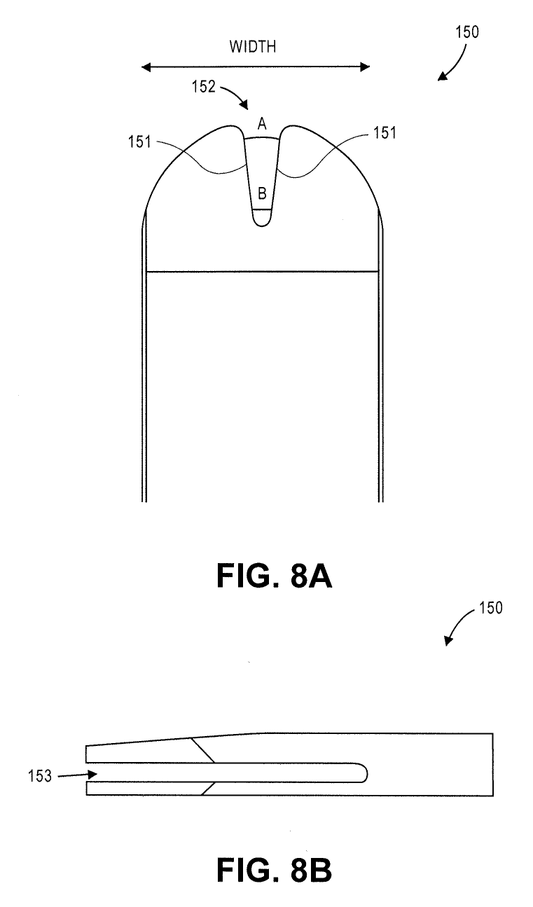

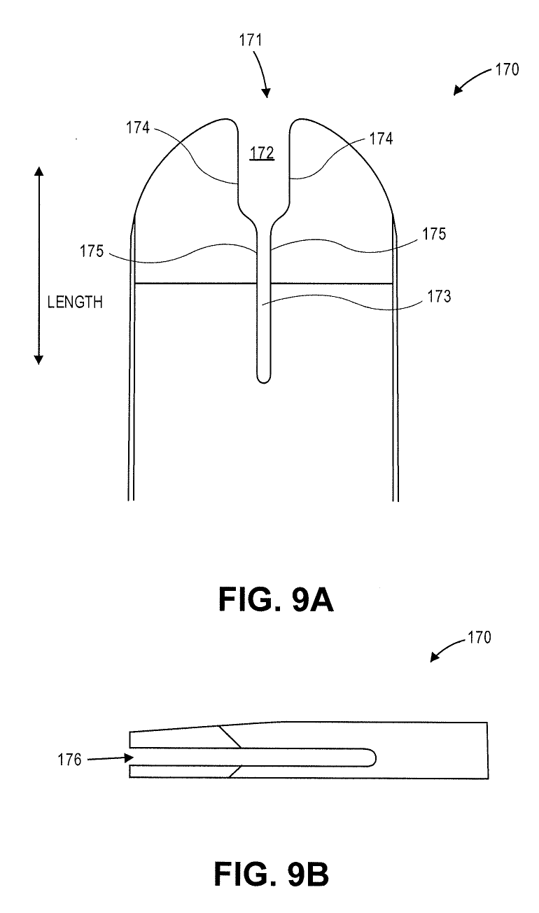

FIGS. 8A and 8B (top and side views, respectively) illustrate a distal region of an exemplary inserter 150 that has an inserter slot 152 and corneal device receiving space 153. Except as described below, inserter 150 can be the same or incorporated with any other inserter components described herein. Inserter slot 152 has a width (measured in the “Width” direction shown in FIG. 8A) that is not constant along the length of the slot. In this embodiment, the slot width narrows gradually and substantially constantly in the proximal direction, with the slot width at the distal end of the slot being greater than the slot width at the proximal end of the slot. Surfaces 151 of the inserter provide the tapered configuration to slot 152. The slot may extend proximally beyond the corneal device receiving area, or it may not extend to the proximal end of the device receiving area. An exemplary advantage of a slot with a tapered configuration (narrowing in the proximal direction), such as shown in FIG. 8A, is that it increases the exposed area at the distal end (via the increased width) to ensure the corneal device is released from the device receiving space, while also improving device retention during shipping (via the narrower proximal end). In other words, the slot configuration can help stabilize the device in the device receiving space due to the narrower proximal portion of the slot, but can help release the device from the corneal device receiving area due to the wider distal portion. The suitable description of any other inserters herein can similarly apply to FIGS. 9A and 9B.

The embodiment in FIGS. 8A and 8B is an example of an inserter with an inserter slot, is an example of an inserter with an inserter slot extending through at least one of top and bottom surface of a distal region of the inserter, is an example of an inserter with an inserter slot, wherein at least a portion of the inserter slot can be proximal to, or behind, a proximal end of an inserter device receiving space, is an example of an inserter with an inserter slot in fluidic communication with an inserter device receiving space, is an example of an inserter with an inserter slot that does not have a constant width along substantially its entire length, and is an example of an inserter slot that extends all the way to an outer edge of a distal region of the inserter.

FIGS. 9A and 9B (top and side views, respectively) illustrate a distal region of exemplary inserter 170, which includes slot 171 that extends from an outer edge of the distal region (the distal end) to a location proximal to device receiving space 176. Inserter slot 171 includes a distal section 172 defined by walls 174 that is wider than a narrower proximal section 173 defined by walls 175. The relatively long length of narrower proximal section 173 of slot 171 (longer than distal section 172) helps direct fluid towards the device to help deploy the device out of the inserter (when the inserter is retracted during deployment), and the narrower width helps retain the device within the device receiving prior to use (e.g., during shipping). The wider distal section 172 of slot 171 increases the exposed area at the distal end (via the increased width) to help release the device from the device receiving space. The suitable description of any other inserters herein can similarly apply to FIGS. 9A and 9B.

The embodiment in FIGS. 9A and 9B is an example of an inserter with an inserter slot, is an example of an inserter with an inserter slot extending through at least one of top and bottom surface of a distal region of the inserter, is an example of an inserter with an inserter slot, wherein at least a least a portion of the inserter slot is proximal to, or behind, a proximal end of an inserter device receiving space, is an example of an inserter with an inserter slot in fluidic communication with an inserter device receiving space, is an example of an inserter with an inserter slot that does not have a constant width along its entire length, and is an example of an inserter slot that extends all the way to an outer edge of a distal region of the inserter.

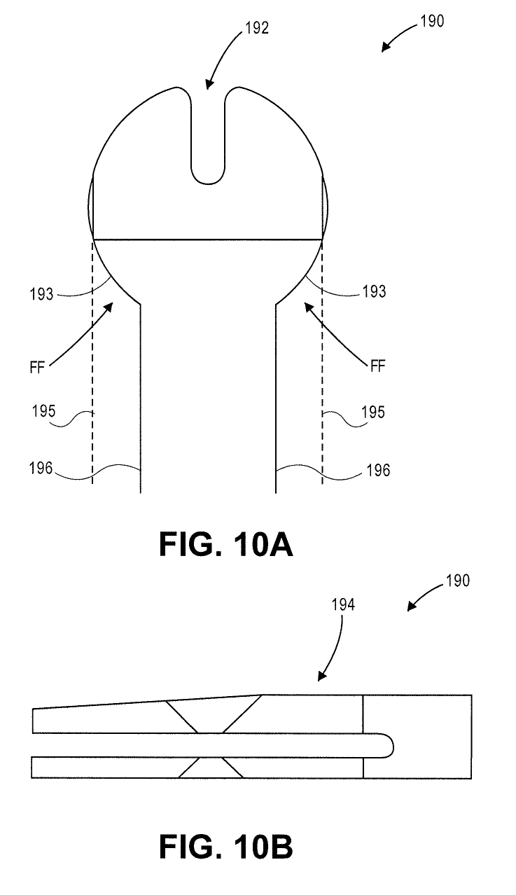

FIGS. 10A and 10B illustrate a distal region of exemplary inserter 190, which is similar to inserter 100 in FIGS. 6A-6C. Inserter 190 does not include a proximal slot such as proximal slot 104 in inserter 100. An additional difference between inserters 190 and an inserter 100 is that inserter 190 has a width that decreases in the proximal direction at the proximal end of the device receiving space. Outer walls 193 and 196 of inserter 190 illustrate the decrease in width of the inserter, compared to the phantom lines 195 indicating the outer walls of inserter 100 from FIGS. 6A-6C. The narrowing proximal section 194 of the inserter exposes the proximal region of the device receiving space to the direction of fluid flow “FF” during the pocket procedure to help release the device from the device receiving space. The suitable description of any other inserters herein can similarly apply to FIGS. 10A and 10B.

The embodiment in FIGS. 10A and 10B is an example of an inserter with an inserter slot, is an example of an inserter with an inserter slot extending through at least one of top and bottom surface of a distal region of the inserter, is an example of an inserter with an inserter slot, wherein at least a least a portion of the inserter slot can proximal to, or behind, a proximal end of an inserter device receiving space, is an example of an inserter with an inserter slot in fluidic communication with an inserter device receiving space, is an example of an inserter with an inserter slot that has a constant width along substantially its entire length, and is an example of an inserter slot that extends all the way to an outer edge of a distal region of the inserter.

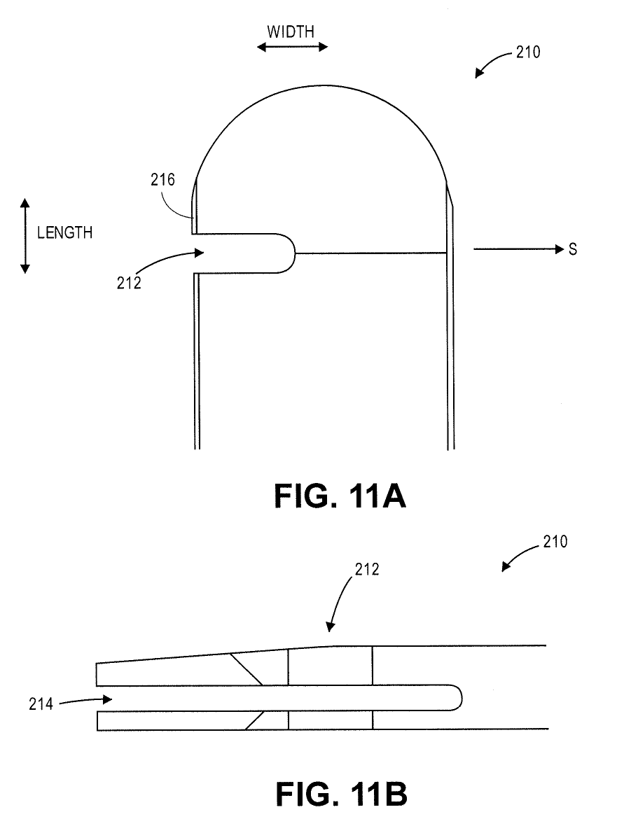

FIGS. 11A and 11B (top and side views, respectively) illustrate a distal region of exemplary inserter 210, which includes slot 212 that extends to an outer edge of the inserter, and is in fluidic communication with device receiving space 214. In this embodiment slot 212 extends to a side wall 216 of the inserter. Slot 212 could alternatively be on the other side of the inserter. Inserter 210 can have additional slots, such as a slot on the other side of the inserter extending to the other sidewall, or any of other types of slots herein. The side slot 212 can help release the device from the device receiving area in response to a sweeping motion in the “S” direction, as shown in FIG. 11A. Slot 212 can also be used in a flap procedure, described in applications incorporated by reference and known in the art (as can all of the other inserters herein even if it is not specifically mentioned in the description of those inserters), and, if necessary, allows a tool to be positioned in the side slot to remove the device from the inserter. The suitable description of any other inserters herein can similarly apply to FIGS. 11A and 11B.

The embodiment in FIGS. 11A and 11B is an example of an inserter with an inserter slot, is an example of an inserter with an inserter slot extending through at least one of top and bottom surface of a distal region of the inserter, is an example of an inserter with an inserter slot in fluidic communication with an inserter device receiving space, is an example of an inserter with an inserter slot that has a constant length along substantially its entire width, and is an example of an inserter slot that extends all the way to an outer edge of a distal region of the inserter.

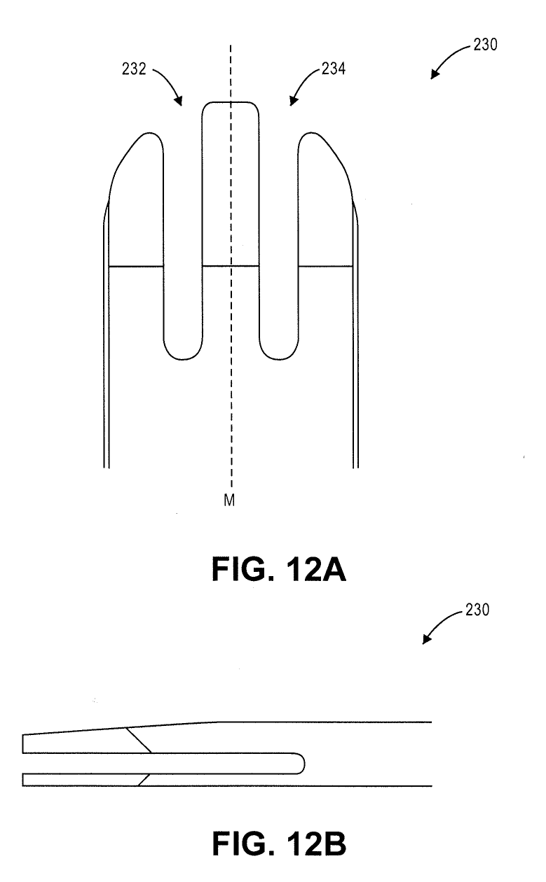

FIGS. 12A and 12B (top and side views, respectively) illustrate a distal region of exemplary inserter 230. Inserter 230 includes first slot 232 and second slot 234, on different sides of midline M of the inserter. In this embodiment the inserter is symmetrical about midline M, but the slots can also have different configurations and the inserter need not be symmetrical about midline. Inserter 230 can be modified to have more than two slots, such as a slot at the midline, which can have a different configuration that either or both of slots 232 and 234. The plurality of slots increases the total cross-sectional area of the anterior and posterior surfaces of the corneal device that are exposed to corneal tissue and fluid (not covered by inserter material), yet by having a plurality of slots, the exposed area is distributed between the two slot zones, which can help retain the device in the corneal device receiving area prior to use (e.g., during shipping). The inserter could have a plurality of slots on one or more sides of the inserter. The suitable description of any other inserters herein can similarly apply to FIGS. 12A and 12B.

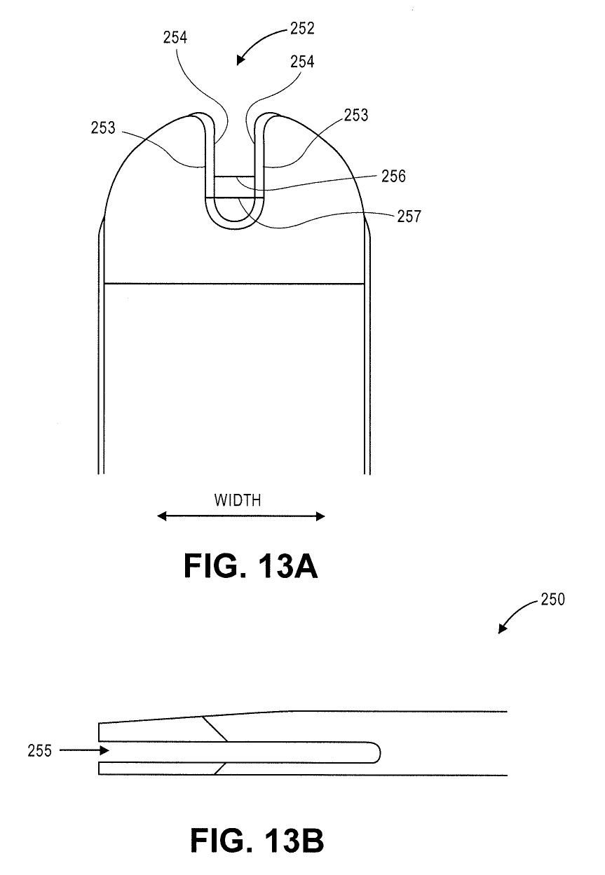

FIGS. 13A and 13B (top and side views, respectively) illustrate a distal region of exemplary inserter 250 that includes a non-symmetrical slot 252. The slot at the top portion of the inserter (above the device receiving area), defined by walls 253 of the extensions, has a first width 257 that is different (in this case greater) than width 256 of the slot at the bottom portion of the inserter (below the device receiving area), defined by walls 254 of the extensions. The slot in the top portion is also longer than the slot in the bottom portion. In other words, the portion of the slot above the corneal device receiving space has a different size and/or configuration that the portion of the slot below the corneal device receiving space. An exemplary advantage of the different slot sizes (top vs bottom) is to increase the exposed area of the device either on the anterior side or the posterior side relative to the other side of the device. For example, the slot on the top in this embodiment would expose more area of the anterior surface of the device than the posterior surface. Corneal device receiving area 255 is shown in FIG. 13B. The suitable description of any other inserters herein can similarly apply to FIGS. 13A and 13B.

The embodiment in FIGS. 13A and 13B is an example of an inserter with an inserter slot, is an example of an inserter with an inserter slot extending through at least one of top and bottom surface of a distal region of the inserter, is an example of an inserter with an inserter slot, wherein at least a least a portion of the inserter slot could be proximal to, or behind, a proximal end of an inserter device receiving space, is an example of an inserter with an inserter slot in fluidic communication with an inserter device receiving space, is an example of an inserter with an inserter slot that has a constant width along its length (both the top and bottom portions of the slot), is an example of an inserter slot that extends all the way to an outer edge of a distal region of the inserter, and is an example of an inserter slot for which at least one of the configuration and the size of the top and bottom slot portions is different.

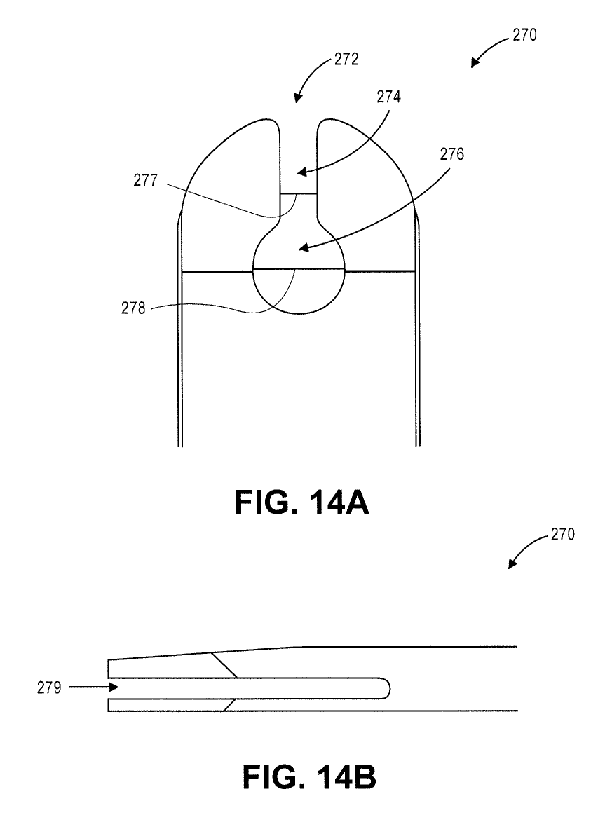

FIGS. 14A and 14B (top and side views, respectively) illustrate a distal region of exemplary inserter 270 with slot 272, wherein slot 272 includes distal section 274 and proximal section 276, wherein proximal section 276 (which has a distal end where slot 272 begins to widen relative to distal section 274) comprises a greater cross sectional area that distal section 272. Distal slot section 274 has a constant width over substantially its entire length, and proximal slot section 276 is generally circular. This slot configuration increases the cross-sectional area of the slot in the proximal region of the slot. This can help direct fluid forward towards the distal end of the inserter and help release the device from the corneal device receiving area as the inserter is retracted during a pocket delivery procedure. The suitable description of any other inserters herein can similarly apply to FIGS. 14A and 14B.

Additionally, the material of the inserter and/or surface roughness, of at least the distal region, can also influence the delivery of the device. For example, one or more coatings on one or more portions of the distal region can aid in the delivery of the inserter into, and/or removal from, a pocket.

The inserter and devices described in exemplary FIGS. 1A-14B can be adapted to maintain the device in the inserter receiving area with passive forces, such as surface adhesion of the device to the inserter. In alternative embodiments, however, the distal geometry could be modified to comprise a forceps-type instrument that could be actuated to provide active “squeezing” forces during the insertion phase, and release of these active forces during delivery and removal of the device. For example, any aspect of a forceps-type device such as described in U.S. Pub. No. 2011/0172675 can be incorporated into any of the embodiments or concepts described herein.

Any corneal devices can be used, regardless of size, configuration, and material. For example, corneal devices that may be inserted or positioned with any inserter herein can have a shape, dimensions, and be made of materials other than those descried herein, such as disc-shaped, ovoid-shaped, spherical, non-hydrogel, for presbyopia, for myopia, for hyperopia, etc. The inserter body may need to be modified (e.g., one or more dimensions), however, from those embodiments herein, to accommodate a particular device.

As used herein, “slot” generally refers to an inserter slot, and is different than an device slot, or device receiving slot.

The disclosure also includes the following additional embodiments:

### Additional Embodiment 1

A corneal device inserter, comprising: an elongate distal region comprising a corneal device receiving space, and a slot.

### Additional Embodiment 2

A corneal device inserter, comprising: an elongate distal region comprising a corneal device receiving space and a slot extending along at least a portion of the distal region.

### Additional Embodiment 3

A corneal device inserter, comprising: an elongate distal region comprising a corneal device receiving space and slot extending through at least one of top and bottom surfaces of the distal region.

### Additional Embodiment 4

A corneal device inserter, comprising: an elongate distal region comprising a corneal device receiving space and a slot, at least a portion of the slot being proximal to, or behind, the corneal device receiving space.

### Additional Embodiment 5

A corneal device inserter, comprising: an elongate distal region comprising a corneal device receiving space and a slot, the corneal device receiving space and the slot being in fluidic communication.

### Additional Embodiment 6

The inserter of any of the additional embodiments, wherein the slot defines first and second arms on either side of the slot.

### Additional Embodiment 7

The inserter of any of the additional embodiments, wherein the slot extends through the midline of the elongate distal region.

### Additional Embodiment 8

The inserter of any of the additional embodiments, wherein the corneal device receiving space is at least partially defined by a top surface and a bottom surface.

### Additional Embodiment 9

The inserter of any of the additional embodiments, wherein the corneal device receiving area includes device slots formed in first and second arms, the two arms at least partially defining the device slot.

### Additional Embodiment 10

The inserter of any of the additional embodiments, wherein the slot has any length described herein.

### Additional Embodiment 11

The inserter of any of the additional embodiments, wherein the slot has any width described herein.

### Additional Embodiment 12

The inserter of any of the additional embodiments, wherein the slot has any height described herein.

### Additional Embodiment 13

The inserter of any of the additional embodiments, wherein the distal region includes a top surface, a bottom surface, and two side walls.

### Additional Embodiment 14

The inserter of any of the additional embodiments, wherein the corneal device receiving area includes an device slot is the distal region.

### Additional Embodiment 15

The inserter of any of the additional embodiments, wherein the slot extends through both a top and a bottom surface of the distal region.

### Additional Embodiment 16

The inserter of any of the additional embodiments, wherein the slot extends through at least one of a top and bottom surface of the distal region.

### Additional Embodiment 17

The inserter of any of the additional embodiments, wherein the slot has a constant width over a substantial portion of its length.

### Additional Embodiment 18

The inserter of any of the additional embodiments, wherein the slot does not have a constant width over a substantial portion of its length.

### Additional Embodiment 19

The inserter of any of the additional embodiments, further comprising a corneal device positioned at least partially within the corneal device receiving space, optionally in a substantially non-deformed configuration.

### Additional Embodiment 19.5

The inserter of any of the additional embodiments, further comprising a corneal device positioned completely within the boundary of the corneal device receiving space.

### Additional Embodiment 20

The inserter of any of the additional embodiments, further comprising a corneal device comprising a hydrogel.

### Additional Embodiment 21

The inserter of any of the additional embodiments, further comprising a corneal device with a water content at least 50%.

### Additional Embodiment 22

The inserter of any of the additional embodiments, further comprising a corneal device with a meniscus configuration or a pinhole configuration.

### Additional Embodiment 23

An inserter with a distal region not adapted to be reconfigured, the inserter adapted to deliver a corneal device within a corneal pocket without an active fluid delivery step.

### Additional Embodiment 24

An inserter with a distal region not adapted to be reconfigured, the inserter adapted to deliver a corneal device within a corneal pocket without an active fluid delivery step and without the need for a separate tool, such as a Sinskey hook.

### Additional Embodiment 25

An inserter with a slot in at least a portion of a distal region, the slot having any combination of length, width, and height described herein.

### Additional Embodiment 26

A corneal device inserter, comprising: an inserter slot through at least one of a top and a bottom surface of a distal region of the corneal device inserter; at least one device receiving slot in communication with a distal end of the inserter and in communication with the inserter slot, wherein at least a portion of the inserter slot is proximal to the at least one device receiving slot.

### Additional Embodiment 27

A corneal device inserter, comprising: a distal region including a corneal device slot that extends from a first side to a second side of the distal region, and a space in communication with the corneal device slot that extends further proximally than the corneal device slot, wherein the space does not extend to the first side and the second side of the distal region.

### Additional Embodiment 28

A corneal device inserter comprising a space proximal relative to an device receiving space in a distal region of the inserter.

### Additional Embodiment 29

Any of the additional embodiment inserters, wherein a top extension and a bottom extension, in a top view, have the same configuration and dimensions and thus overlap one another.

### Additional Embodiment 30

Any of the additional embodiment inserters, wherein the inserter slot includes a proximal-most surface, the proximal most surface extending from a bottom surface of the distal region to a top surface of the distal region.

### Additional Embodiment 31

A method of releasing a corneal device from an inserter, wherein releasing the corneal device does not require active fluid delivery, a plunger mechanism, or reconfiguring the distal region of an inserter.

### Additional Embodiment 32

Any of the additional embodiment inserters, wherein a corneal device receiving space has a constant height along a length of the corneal device receiving space (for example, FIG. 1E).

### Additional Embodiment 33

Any of the additional embodiment inserters, wherein the inserter is adapted so that a top surface and a bottom surface of a distal inserter region are adapted to be moved away from each other.

### Additional Embodiment 34

A corneal device inserter, comprising an device receiving space and a plurality of slots or recesses in a distal region, optionally wherein one or more of the slots extends further proximally than the device receiving space.

### Additional Embodiment 35

Any of the additional embodiments inserters, wherein the inserter comprises first and second slots, optionally wherein one slot is a window slot that does not extend to an outer (i.e., side) edge of the inserter, in a top view.

### Additional Embodiment 36

Any of the additional embodiment inserters, wherein a surface of the slot is beveled.

### Additional Embodiment 37

Any of the additional embodiment inserters, wherein the slot does not have a constant width over its entire length, such as a slot that tapers in the proximal direction.

### Additional Embodiment 38

Any of the additional embodiment inserters, wherein the slot comprises a first section and a second section, the first section having a width different than a width of the second section.

### Additional Embodiment 39

Any of the additional embodiment inserters, wherein an inserter side, or edge, wall narrows in the region of the proximal portion of a corneal device receiving space to expose a proximal region of the corneal device receiving space.

### Additional Embodiment 40

Any of the additional embodiment inserters, wherein the slot extends to the edge of a side wall of the inserter.

### Additional Embodiment 41

Any of the additional embodiment inserters, comprising a plurality of slots, optionally a first of which is on one side of a midline of the inserter and a second slot is on a second side of the midline.

### Additional Embodiment 42

Any of the additional embodiment inserters, comprising a nonsymmetrical slot, optionally nonsymmetrical about a midline of the inserter.

### Additional Embodiment 43

A method of releasing a corneal device from an inserter into a corneal pocket, comprising: retracting the inserter proximally out of the pocket, wherein retracting the inserter proximally causes the device to be released from the inserter and deposited in the pocket.

### Additional Embodiment 44

The method of additional embodiments 43 wherein the method does not require active fluid delivery, reconfiguring a distal region of the inserter, or using a separate tool, to release the device from the inserter.

1. A corneal device inserter, comprising:
an elongate member comprising a distal region, the distal region including a top portion, a bottom portion, and a corneal device receiving space, the corneal device receiving space defined at least partially by the top portion, the bottom portion, and a receiving space proximal end,
the distal region further comprising a slot in fluid communication with the corneal device receiving space,
the slot extending through at least one of the top portion and the bottom portion, the slot having a slot proximal end disposed at least as far proximally as the receiving space proximal end.

2. The inserter of claim 1, wherein the slot proximal end is disposed proximal to the receiving space proximal end.

3. The inserter of claim 1, wherein the slot does not extend to a distal end of the distal region.

4. The inserter of claim 1, wherein the distal region comprises a second slot extending through at least one of the top portion and the bottom portion and in fluid communication with the corneal device receiving space, the second slot extending to the distal end of the distal region.

5. The inserter of claim 1, wherein the slot extends to a distal end of the distal region.

6. The inserter of claim 1, wherein the slot is symmetric about a distal region longitudinal axis that extends along the distal region and through the distal end.

7. The inserter of claim 1, wherein the distal region comprises first and second arms extending to a distal end of the elongate region.

8. The inserter of claim 7 wherein the first and second arms are defined by the slot.

9. The inserter of claim 8 wherein the first and second arms are defined by a second slot different than the slot.

10. The inserter of claim 1 wherein the slot extends through the top portion and the bottom portion.

11. The inserter of claim 1, wherein the slot proximal end is between 0 mm and 5 mm proximal to the receiving space proximal end.

12. The inserter of claim 1, wherein the slot has a length from 0.5 mm and 5 mm.

13. The inserter of claim 12, wherein the slot has a length from 2 mm to 4 mm.

14. The inserter of claim 1, wherein the slot has a greatest width dimension from 0.2 mm to 2 mm.

15. The inserter of claim 14, where the slot has a greatest width dimension from 0.2 mm to 0.7 mm

16. The inserter of claim 1, wherein a width of the slot relative to an overall width of a distal region of the inserter is from 5% to 40%, optionally 10% to 30%, such as 20%.

17. The inserter of claim 1, wherein a distance between a distal end of the slot and a proximal end of the receiving space is from 0.1 mm to 0.5 mm, optionally 0.2 mm to 0.5 mm, optionally 0.3 mm to 0.45 mm.

18. The inserter of claim 1, wherein a distance from a distal end of the inserter to a distal end of the slot is from 0.5 mm to 2.5 mm, optionally 1 mm to 2.25 mm.

19. The inserter of claim 1, wherein a length of a the slot is from 0.2 mm to 1.8 mm, optionally 0.4 mm to 1.5 mm, optionally 0.5 mm to 1.3 mm.

20. The inserter of claim 1, wherein a width of a the slot is from 0.2 mm to 1.5 mm, optionally 0.2 mm to 1 mm, optionally 0.2 mm to 0.7 mm.

21. The inserter of claim 1, wherein a length of a bridge between the slot and a distal slot is 0.2 mm to 1.2 mm, such as 0.5 mm to 1 mm.

22. The inserter of claim 1, wherein a height of the receiving space is 0.075 mm to 0.225 mm, such as 0.1 mm to 0.2 mm.

23. The inserter of claim 1, wherein a length of the receiving space, is from 1.25 mm to 3.5 mm, such as 1.5 mm to 3 mm.

24. The inserter of claim 1, wherein the slot has a uniform width over at least 70% of its length.

25. The inserter of claim 1, wherein the slot does not have a uniform width over at least 70% of its length.

26. The inserter of claim 1, further comprising a corneal device positioned at least partially within the corneal device receiving space, optionally in a substantially non-deformed configuration.

27. The inserter of claim 26, wherein the corneal device is selected from the group consisting of a corneal device with a meniscus configuration and a corneal device with a pinhole configuration.

28. The inserter of claim 1, wherein the distal region is not adapted to be reconfigured.

29. The inserter of claim 1, wherein the slot and receiving space are sized and positioned such that a corneal device can be delivered into a corneal pocket from the receiving space by proximally withdrawing the inserter, and without an active fluid delivery step and without the need for a separate delivery tool, such as a Sinskey hook.

30. The inserter of claim 1, wherein the receiving space extends from a first side of the distal region to a second side of the distal region.

31. The inserter of claim 1, wherein a surface of the slot, optionally a distal surface, is beveled.

32. The inserter or claim 1, wherein the slot comprises a first section and a second section, the first section having a width different than a width of the second section.

33. The inserter of claim 1, further comprising a corneal device disposed within the receiving space, optionally in an unstressed configuration.

34. The inserter of claim 33, wherein the corneal device has a water content of at least 50%, such as at least 70%.

35. A method of releasing a corneal device onto corneal tissue, comprising:
positioning an inserter adjacent corneal tissue, optionally within a corneal pocket, wherein the inserter comprises a distal region, the distal region including a top portion, a bottom portion, and a corneal device receiving space, the corneal device receiving space defined by the top portion, the bottom portion, and a receiving space proximal end, the distal region further comprising a slot in fluid communication with the corneal device receiving space, the slot extending through at least one of the top portion and the bottom portion; and
proximally retracting the inserter to release a corneal device from the receiving space,
wherein the method does not require an active fluid delivery step, the use of a plunging mechanism, or reconfiguring the distal region of the inserter to release the corneal device from the receiving space.
